(19) United States
(12) Patent Application Publication
(10) Pub. No.: US 2003/0045473 A1
(43) Pub. Date: Mar. 6, 2003

(54) Compositions, kits, and methods for cardiovascular health

(76) Inventors: Robert Joseph Sarama, Loveland, OH (US); Raymond Louis Niehoff, West Chester, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/181,656

(22) PCT Filed: Jan. 25, 2001
(86) PCT No.: PCT/US01/02382

(51) Int. Cl. A61K 38/16; A61K 31/56; A61K 31/225; A61K 31/198

(52) U.S. Cl. 514/12; 514/171; 514/547; 514/565

(57) ABSTRACT

The present invention is directed to compositions comprising: (a) a first component selected from the group consisting of L-arginine, polypeptides thereof, acceptable salts thereof, pro-forms thereof, and mixtures thereof; and (b) a second component selected from the group consisting of sterols, stanols, esters thereof, polyol fatty acid polyesters, and mixtures thereof. The present invention is further directed to kits comprising these compositions as well as methods of using the compositions. The compositions, kits, and methods herein are useful for providing general health benefits to the consumer, particularly cardiovascular benefits, anti-menopausal benefits and/or treating sexual dysfunction (particularly, erectile dysfunction). Most particularly, the compositions, kits, and methods herein are useful for providing cardiovascular benefits, including lowering cholesterol in the consumer, treating, preventing, and/or inhibiting heart disease (e.g. atherosclerosis, restenosis, thrombosis) and, for example, treating other conditions such as hypercholesterolemia, hypertension, poor circulation, and complications associated with diabetes.

### REFERENCE TO PRIORITY APPLICATION

The present invention claims priority to U.S. Provisional Application Serial No. 60/178,778, filed Jan. 28, 2000.

### FIELD OF THE INVENTION

The present invention relates to compositions, kits, and methods which are useful for providing various general health benefits including, but not limited to cardiac benefits, including lowering cholesterol in the consumer, treating, preventing, and/or inhibiting heart disease (e.g., atherosclerosis, restenosis, thrombosis) and treating conditions such as hypercholesterolemia, hypertension, poor circulation, and complications associated with diabetes.

### BACKGROUND OF THE INVENTION

Cardiovascular conditions, including heart disease, hypercholesterolemia, hypertension, poor circulation, and complications associated with diabetes, are serious medical conditions which are leading causes of mortality in humans. Various regimens have been suggested for prevention and treatment of these conditions, including pharmaceutical, dietary, and exercise regimens. Notwithstanding, they remain among the most prevalent and serious of all medical conditions.

L-arginine is a natural amino acid which has been identified to provide certain general health benefits including, for example, cardiovascular benefits, such as lowering cholesterol in the consumer, and treating, preventing, and/or inhibiting heart disease and poor circulation. See e.g., Moskowitz, U.S. Pat. No. 5,385,940, assigned to The General Hospital Corp., issued Jan. 31, 1995; Sonaka et al., EP 0,546,796, assigned to Ajinomoto Co., published Jun. 16, 1993; Cotter et al., U.S. Pat. No. 4,920,098, assigned to Baxter International Inc., issued Apr. 24, 1990; Dudrick, U.S. Pat. No. 5,032,608, issued Jul. 16, 1991; Levere et al., U.S. Pat. No. 5,217,997, issued Jun. 8, 1993; Cooke et al., U.S. Pat. No. 5,428,070, assigned to Stanford University, issued Jun. 27, 1995; Chibata et al., U.S. Pat. No. 4,420,432, assigned to Tanabe Seiyaky Co., issued Dec. 13, 1983; Varma et al., U.S. Pat. No. 5,364,884, assigned to Baylor College of Medicine, issued Nov. 15, 1994; and Barbul, U.S. Pat. No. 5,157,022, issued Oct. 20, 1992.

The utility of L-arginine, particularly to advance cardiovascular health, is therefore well known in the art. However, as for any beneficial regimen, compliance must be assured in order to realize the various benefits thereof. Unfortunately, L-arginine and its close derivatives (including salts, polypeptides, and pro-forms) have a strong, bitter, and fishy flavor, making L-arginine generally unacceptable for use. This results in decreased compliance of a regimen involving L-arginine, and the requisite cardiovascular benefits are therefore not realized. Accordingly, to enhance compliance, it would be desirable to provide L-arginine in a form which diminishes and/or removes the unacceptable flavor associated with L-arginine.

Unfortunately, flavor improvement is typically associated with a decrease in the general health benefits of the component which is desired to be delivered. Additionally, because delivery of relatively large amounts of L-arginine is desirable (e.g., about 3 grams to about 10 grams of L-arginine per dose), it becomes increasingly more difficult to mask the strong, bitter, and fishy flavor. Such difficulties manifest themselves in the marketplace, where it is understood that current products containing L-arginine are not acceptable to the consumer due to unacceptable flavor.

The present inventors have surprisingly discovered that the unacceptable flavor of L-arginine is significantly improved through combination with a second component, which is described herein as a sterol, stanol, ester thereof, or a polyol fatty acid polyester. Interestingly, and quite unexpectedly, this second component diminishes and/or removes the unacceptable flavor associated with the L-arginine. Accordingly, such combination is acceptable to consumers which, more importantly, translates into improved regimen compliance and enhanced cardiovascular, and other health, benefits. Additionally, the second component does not decrease the cardiovascular health benefits of the resulting composition, but rather enhances such benefits. For example, sterols, stanols, and their esters have been utilized in food compositions to decrease cholesterol. Similarly, polyol fatty acid polyesters (e.g., sucrose polyesters) add no fat to the composition and may reduce cholesterol, but maintain flavor properties of traditional fat products and, in this case, improve the overall flavor of the composition by diminishing and/or removing the unacceptable flavor of the L-arginine.

The foregoing findings are unexpected relative to the known literature. Accordingly, the present inventors have discovered compositions which provide general health benefits, including cardiovascular benefits. Relative to known products, compliance is improved and/or ensured through use of such compositions because the flavor is acceptable to the consumer. The compositions are easily provided as a pharmaceutical or food product (preferably, a food product) and may be delivered in kit form, wherein the kit has the further advantage of disseminating information to the consumer regarding various health benefits and dose regimens of the composition.

### SUMMARY OF THE INVENTION

The present invention is directed to compositions comprising:

(a) a first component selected from the group consisting of L-arginine, polypeptides thereof, acceptable salts thereof, pro-forms thereof, and mixtures thereof; and

(b) a second component selected from the group consisting of sterols, stanols, esters thereof, polyol fatty acid polyesters, and mixtures thereof.

The present invention is further directed to kits comprising these compositions as well as methods of using the compositions. The compositions, kits, and methods herein are useful for providing general health benefits to the consumer, particularly cardiovascular benefits, anti-menopausal benefits and/or treating sexual dysfunction (particularly, erectile dysfunction). Most particularly, the compositions, kits, and methods herein are useful for providing cardiovascular benefits, including lowering cholesterol in the consumer, treating, preventing, and/or inhibiting heart disease (e.g., atherosclerosis, restenosis, thrombosis) and, for example, treating other conditions such as hypercholesterolemia, hypertension, poor circulation, and complications associated with diabetes.

### DETAILED DESCRIPTION OF THE INVENTION

The present invention is directed to compositions which are useful for providing general health benefits to the consumer, particularly cardiovascular benefits, anti-menopausal benefits and/or treating sexual dysfunction (particularly, erectile dysfunction). The invention herein is further directed to kits comprising the compositions and methods of using the compositions to provide the foregoing general health benefits.

Publications, patents, and patent applications are referred to throughout this disclosure. All references cited herein are hereby incorporated by reference.

All percentages and ratios are calculated by weight unless otherwise indicated. All percentages and ratios are calculated based on the total composition unless otherwise indicated.

All component or composition levels are in reference to the active level of that component or composition, and are exclusive of impurities, for example, residual solvents or by-products, which may be present in commercially available sources.

Referred to herein are trade names for components including, but not limited to, certain carbohydrates, flavors, and other components. The inventors herein do not intend to be limited by materials under a certain trade name. Equivalent materials (e.g., those obtained from a different source under a different name or catalog (reference) number) to those referenced by trade name may be substituted and utilized in the compositions, kits, and methods herein.

In the description of the invention various embodiments and/or individual features are disclosed. As will be apparent to the ordinarily skilled practitioner, all combinations of such embodiments and features are possible and can result in preferred executions of the present invention.

The compositions, methods, and kits herein may comprise, consist essentially of, or consist of any of the elements as described herein.

### Compositions of the Present Invention

The present invention is directed to compositions which are useful for providing general health benefits to the consumer, particularly cardiovascular benefits, anti-menopausal benefits and/or treating sexual dysfunction (particularly, erectile dysfunction). The invention herein is further directed to kits comprising the compositions and methods of using the compositions to provide the foregoing general health benefits. Most particularly, the compositions, kits, and methods herein are useful for providing cardiovascular benefits, including lowering cholesterol in the consumer, treating, preventing, and/or inhibiting heart disease (e.g., atherosclerosis, restenosis, thrombosis) and, for example, treating other conditions such as hypercholesterolemia, hypertension, poor circulation, and complications associated with diabetes.

The compositions herein comprise:

(a) a first component selected from the group consisting of L-arginine, polypeptides thereof, acceptable salts thereof, pro-forms thereof, and mixtures thereof; and

(b) a second component selected from the group consisting of sterols, stanols, esters thereof, polyol fatty acid polyesters, and mixtures thereof.

The present inventors have discovered that such compositions are particularly useful for delivering cardiovascular benefits through a synergistic combination of the L-arginine (including polypeptides, acceptable salts, and pro-forms thereof) and the sterols, stanols, esters thereof, or a polyol fatty acid polyester. As a further beneficial aspect of these compositions, the present inventors have surprisingly discovered that the undesirable flavor of L-arginine is significantly diminished or removed through combination with the second component. This surprising and unexpected results allows for enhanced delivery and compliance associated with ingestion of L-arginine for various health benefits, while additionally providing the health benefits known to be associated with the second component.

As used herein, the first component is selected from L-arginine, polypeptides thereof, acceptable salts thereof, pro-forms thereof, and mixtures thereof. Preferably, the first composition is selected from L-arginine and salts thereof. As further used herein, the second component is selected from sterols, stanols, esters thereof, polyol fatty acid polyesters, and mixtures thereof. The terms “first component” and “second component” are utilized herein strictly for convenience of reference and in no manner are these terms intended to limit order of addition to the composition, importance of the various components, and any other limiting factors.

### First Component

The first component of the present compositions is selected from the group consisting of L-arginine, polypeptides thereof, salts thereof, pro-forms thereof, and mixtures thereof. L-arginine is a natural amino acid which has been identified to provide certain general health benefits including, for example, cardiovascular benefits, including lowering cholesterol in the consumer, and treating, preventing, and/or inhibiting heart disease (e.g., atherosclerosis, restenosis, hypertension, poor circulation, and/or complications associated with diabetes. See e.g., Moskowitz, U.S. Pat. No. 5,385,940, assigned to The General Hospital Corp., issued Jan. 31, 1995; Sonaka et al., EP 0,546,796, assigned to Ajinomoto Co., published Jun. 16, 1993; Cotter et al., U.S. Pat. No. 4,920,098, assigned to Baxter International Inc., issued Apr. 24, 1990; Dudrick, U.S. Pat. No. 5,032,608, issued Jul. 16, 1991; Levere et al., U.S. Pat. No. 5,217,997, issued Jun. 8, 1993; Cooke et al., U.S. Pat. No. 5,428,070, assigned to Stanford University, issued Jun. 27, 1995; Chibata et al., U.S. Pat. No. 4,420,432, assigned to Tanabe Seiyaky Co., issued Dec. 13, 1983; Varma et al., U.S. Pat. No. 5,364,884, assigned to Baylor College of Medicine, issued Nov. 15, 1994; and Barbul, U.S. Pat. No. 5,157,022, issued Oct. 20, 1992.

Wherein L-arginine, a polypeptide thereof, salt thereof, or mixture thereof is utilized in the compositions, typically from about 0.0001% to about 25%, by weight of the composition, is utilized in such composition. More preferably from about 0.1% to about 20%, even more preferably from about 1% to about 15%, and most preferably from about 3% to about 10%, by weight of the composition, is utilized in such composition. Additionally, as a daily dose is frequently important for maintenance of the general health benefits provided by the L-arginine, typically from about 0.05 grams to about 50 grams of the L-arginine, polypeptide thereof, salt thereof, or mixture thereof is administered daily in such composition. More preferably, from about 0.01 grams to about 20 grams, even more preferably from about 0.1 gram to about 10 grams, and most preferably from about 0.5 grams to about 6 grams of the L-arginine, polypeptide thereof, salt thereof, or mixture thereof is administered daily in such composition.

The L-arginine utilized herein as the first component may be used in its free form or may be utilized as a salt, a polypeptide, and/or a pro-form. Salts of L-arginine are particularly preferred herein as they typically provide enhanced palatability relative to the free form of L-arginine. The salt used herein should be an acceptable salt, i.e., a salt useful in pharmaceutical and/or food compositions, preferably food compositions. Many suitable salts of L-arginine are commonly known to one of ordinary skill in the art. For example, Greenberg et al., U.S. Pat. No. 5,780,039, assigned to Novartis Nutrition, issued Jul. 14, 1998 discloses palatable forms of L-arginine as acceptable salts. Such salts include those of food grade acids such as phosphoric, citric, adipic, tartaric, acetic, fumaric, malic, and lactic acid. Thus, as non-limiting examples phosphate, citrate, acetate, malate, tartrate, fumarate, adipate, and lactate salts of L-arginine may be utilized as the first component herein. Additionally, the hydrochloride salt of L-arginine may be similarly utilized. The acetate and hydrochloride salts of L-arginine are particularly preferred.

Polypeptides of L-arginine are also well-known in the art. Preferred polypeptides for use herein include those which are readily hydrolyzed in vivo to provide free L-arginine. Dipeptides and tripeptides of L-arginine are particularly preferred. Pro-forms of L-arginine may also be utilized herein. Pro-forms (also commonly referred to as pro-drugs) are those forms which, upon hydrolysis in vivo, provide the free L-arginine. Non-limiting, but preferred, examples of such pro-forms include the esters and amides of L-arginine, for example, L-arginine methyl, ethyl, propyl, or butyl ester, preferably methyl ester. Amides of the □-nitrogen of L-arginine are also particularly useful as pro-forms herein.

### Second Component

The second component of the present compositions is selected from sterols, stanols, esters thereof, polyol fatty acid polyesters, and mixtures thereof. By “esters thereof” it is meant that sterol esters and stanol esters are included within the definition of the second component.

Sterols, Stanols, and Esters Thereof

The second component may be a sterol, stanol, ester thereof, or mixtures thereof. Such sterols, stanols, and esters have recently been identified as useful for certain cardiovascular benefits, including lowering cholesterol. The present inventors have surprisingly discovered that combination of such sterols, stanols, and/or esters with the first component herein provides several unexpected benefits. For example, the sterols, stanols, and/or esters (particularly wherein esters are included) encapsulate the first component to provide sustained delivery of the first component. Additionally, the combination also diminishes and/or removes the adverse flavor typically associated with the first component. As a further advantage, the sterols, stanols, and/or esters interact synergistically with the first component to provide the foregoing cardiovascular benefits.

In a particularly preferred embodiment herein, the present compositions comprise a mixture of at least one sterol or stanol and at least one sterol ester or stanol ester. In a more preferred embodiment of the present invention, the composition comprises a mixture selected from: a) a mixture of one or more sterols and one or more sterol esters; and b) a mixture of one or more stanols and one or more stanol esters. In the most preferred embodiment herein, the composition comprises a mixture selected from: a) a mixture of one or more sterols and one or more sterol fatty acid esters; and b) a mixture of one or more stanols and one or more stanol fatty acid esters. Without intending to be limited by theory, the present inventors have discovered that the foregoing mixtures encapsulate (coat) the first component which further disguises the adverse flavor of the first component when administered orally. Additionally, sustained delivery of the first component is accomplished through such encapsulation, providing enhanced and prolonged bioavailability of the first component to provide the requisite health benefits.

Wherein a mixture of at least one sterol or stanol and at least one sterol ester or stanol ester is utilized, the ratio of the sterols/stanols relative to the sterol/stanol esters can be important. Preferably, the ratio of sterols and stanols to the esters is from about 99:1 to about 1:99. More preferably, the ratio of sterols and stanols to the esters is from about 75:25 to about 25:75. Most preferably, the ratio of sterols and stanols to the esters is from about 60:40 to about 40:60. In all of the foregoing, such ratios are calculated by weight of the sterols, stanols, and esters.

Wherein a sterol, stanol, ester thereof, or mixture thereof is utilized in the compositions, typically from about 0.0001% to about 25%, by weight of the composition, is utilized in such composition. More preferably from about 0.1% to about 20%, even more preferably from about 1% to about 15%, and most preferably from about 3% to about 10%, by weight of the composition, is utilized in such composition. Additionally, as a daily dose is frequently important for maintenance of the general health benefits provided by the sterol, stanol, or ester, typically from about 0.01 grams to about 50 grams of the sterol, stanol, ester, or mixture thereof is administered daily. More preferably, from about 0.05 grams to about 20 grams, even more preferably from about 0.1 gram to about 6 grams, and most preferably from about 0.2 grams to about 4 grams of the sterol, stanol, ester, or mixture thereof is administered daily.

Sterols, stanols, and esters thereof (particularly fatty acid esters thereof), which are useful as the second component herein, are commonly known in the art. As non-limiting examples, such second components are described in Stern, U.S. Pat. No. 3,004,043, assigned to Eastman Kodak Co., issued Oct. 10, 1961; Wruble et al., U.S. Pat. No. 3,085,939, issued Apr. 1, 1963; Erickson, U.S. Pat. No. 3,751,569, assigned to The Procter & Gamble Co., issued Aug. 7, 1973; Jandacek, U.S. Pat. No. 3,865,939, assigned to The Procter & Gamble Co., issued Feb. 11, 1975; Ong, U.S. Pat. No. 4,195,084, assigned to Eli Lilly and Co., issued Mar. 25, 1980; Malinow, U.S. Pat. No. 4,461,762, assigned to Medical Research Foundation, issued Jul. 24, 1984; Arichi et al., U.S. Pat. No. 4,524,067, assigned to Osaka Chemical Lab. Co., issued Jun. 18, 1985; Malinow, U.S. Pat. No. 4,602,003, assigned to Medical Research Foundation, issued Jul. 22, 1986; Cassal, U.S. Pat. No. 4,680,290, assigned to Hoffman-La Roche Inc., issued Jul. 14, 1987; Ambrus et al., U.S. Pat. No. 5,112,815, issued May 12, 1992; Straub, U.S. Pat. No. 5,244,887, issued Sep. 14, 1993; Eugster et al., U.S. Pat. No. 5,270,041, assigned to Marigen S.A., issued Dec. 14, 1993, Mazur et al., U.S. Pat. No. 5,591,836, assigned to The Procter & Gamble Co., issued Jan. 7, 1997; Moreau et al., U.S. Pat. No. 5,843,499, assigned to United States of America, issued Dec. 1, 1998; Miettenen et al., U.S. Pat. No. 5,958,913, assigned to Raisio Benecol Ltd., issued Sep. 28, 1999; Karppanen et al., WO 98/28990, assigned to Pharmaconsult, published Jul. 9, 1998; Shirakawa et al., EP 0,289,636, published Nov. 9, 1988; Ko, WO 94/18225, assigned to Du Pont Merck Pharmaceutical, published Aug. 18, 1994; Festo, WO 95/08342, assigned to Inpharma S.A., published Mar. 30, 1995; Ritter et al., WO 97/42830, assigned to Unilever PLC, published Nov. 20, 1997; Van Amerongen et al., WO 98/01126, assigned to Unilever PLC, published Jan. 15, 1998; and Wester et al., WO 98/06405, assigned to Raision Tehtaat, published Feb. 19, 1998. Any of the sterols and stanols described in the foregoing references, as well as those commonly known in the art, may be included within the second component of the present compositions.

Thus, the term “sterol” as used herein can include natural or synthetic plant or animal sterols or triterpenes. This includes the phytosterols and the mycosterols as well as cholesterol, however it is preferred herein that cholesterol itself is not utilized. For a more detailed discussion of sterols see, for example, Nes, W. D., Parish, E. J., Eds., “Analysis of Sterols and Other Biologically Significant Steroids”, Academic Press, Inc. (1989). Non-limiting examples of preferred sterols include diosgenin, stigmastanol, tigogenin, □-sitosterol, □-sitosterol, stigmasterol, ergosterol, campesterol, oleanoic acids, soyasapogenols, protoascigenin, togenols, protoparaxadiols, protopanaxadiols, □-amyrin, □-amyrin, lupeol, butyrospermol, germanicol, 4-desmethylsterols, 4-monomethylsterols, and 4,4′-dimethylsterols. Other non-limiting examples of sterols for use herein include 7-dehydrocholesterol, 22-dehydrocholesterol, 24-dehydrocholesterol, zymosterol, □⁷-cholesterol, cerebrosterol, 22-□-oxycholesterol, 22-dihydroerogosterol, neospongosterol, cerebisterol, corbisterol, focosterol, □-spinasterol, sargasterol, 7-dehydrocryonasterol, poriferasterol, chondrillasterol, cryonasterol (□-sitosterol), dihydro-□-sitosterol, 14-dehydroergosterol, 24(28)-dehydroergosterol, ergosterol, brassicasterol, 24-methylenecholesterol, ascosterol, episterol, fecosterol, and 5-dihydroergosterol.

It is particularly preferred herein that phytosterols, the stanols derived therefrom (referred to herein as phytostanols), and esters thereof are utilized herein. The term phytosterol is intended to mean unsaturated sterol alcohols and their mixtures derived from plants, as well as synthetically produced sterol alcohols and their mixtures which are either identical to those sterols found in nature, or having properties which are similar to those of naturally occurring sterols. As is well-known in the art, phytosterols (also commonly referred to as plant sterols) are natural components of, for example, vegetable fats and oils. As is also commonly understood, the saturated forms of these sterols (i.e., the forms derived therefrom) are stanols.

The most preferred phytosterols for use as the second component herein include sitosterol (e.g., □-sitosterol (24-ethyl-5□-cholestane-3□-ol) and 5□-sitosterols), stigmasterol, and campesterol. Schematic drawings of these components are as given in S. P. Kochhar, “Influence of Processing on Sterols of Edible Vegetable Oils”, Prog. Lipid Res., Vol. 22, pp. 161-188. For example, □-sitosterol has the following structure:

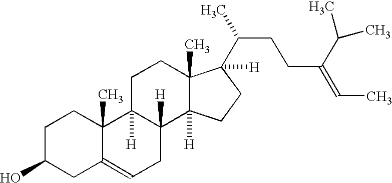

Preparation of such phytosterols is commonly known; for example, sitosterol can be obtained from wood and from refining vegetable oil, and normally comprises also a minor amount of other sterols, such as campesterol, stigmasterol, and various avenasterols. Other suitable phytosterols for use herein include brassicasterol and 22,23-dihydrobrassicasterol.

As described herein above, one or more stanols may be utilized as the second component of the present compositions. Stanols are found in small amounts in nature in such products as wheat, rye, corn, and triticale. They can also easily be produced by hydrogenation of natural sterol mixtures such as vegetable oil-based sterol mixtures or commercially available wood sterols. The plant sterols thus obtained can be converted into stanols by well-known hydrogenation techniques such as those based on the use of a Pd/C catalyst (or other similar catalyst) in organic solvent. A wide variety of palladium catalysts and solvents are known to those of ordinary skill in the art and such catalysis can be used to hydrogenate the sterol for formation of the desired stanol. For example, □-sitostanol (24-ethyl-5□-cholestane-3□-ol) may be prepared by hydrogenation of □-sitosterol in organic solvent.

Accordingly, any sterol, including the foregoing examples of sterols, may be utilized to provide the desired stanol. Accordingly, non-limiting examples of useful stanols include the hydrogenation products of the sterols described herein. The most preferred stanols herein include stanols of the phytosterols, for example, sitostanols (e.g., □-sitostanol and 5□-sitostanols), campestanol, 24-□-methyl cholestanol, stigmastanol, clionastanol, and dihydrobrassicastanol. For example, four major plant stanols are campestanol, 22,23-dihydrobrassicastanol, □-sitostanol, and clionastanol, which have the following structure:

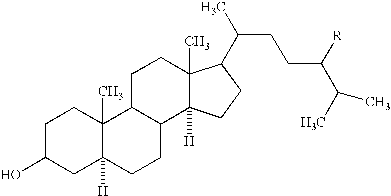

wherein R is —CH₃ for campestanol and its epimer, 22,23-dihydrobrassicastanol and wherein R is —C₂H₅ for sitostanol and its epimer, clionastanol. Campestanol and 22,23-dihydrobrassicastanol differ only by their steric configuration at C₂₄. Similarly, sitostanol and clionastanol differ only by their steric configuration at C₂₄. Alternate nomenclature for clionastanol is (3□, 5□, 24S)-stigmast-5an-3-ol; sitostanol is (3□, 5□, 24R)-stigmast-5an-3-ol; campestanol is (3□, 5□, 24R)-ergost-5an-3-ol; dihydrobrassicastanol is (3□, 5□, 24S)-ergost-5an-3-ol.

It is further understood by one of ordinary skill that sterols, stanols, or their blends, can be utilized to produce sterol esters and/or stanol esters utilized in the present invention. As described below, such sterol and/or stanol esters are also particularly useful in the compositions of the present invention.

The esters of sterols and stanols are readily prepared by one of ordinary skill in the art. Utilization of sterol and/or stanol esters is particularly preferred herein for encapsulation of the first component. Such encapsulation is particularly useful wherein a sterol and/or stanol ester is used alone or, as particularly preferred, as a mixture with at least one sterol or stanol. It has been discovered that such encapsulation, which is described further herein below, surprisingly diminishes or removes the unpalatable flavor associated with the first component. Additionally, sustained delivery of the first component is accomplished through such encapsulation, providing enhanced and prolonged bioavailability of the first component to provide the requisite health benefits. Furthermore, as stated above, use of the sterol and/or stanol ester provides unique health benefits as well.

The sterols and stanols herein may be esterified by any means utilizing any appropriate precursor, for example, phenolic acids such as ferulic acid, coumaric acid, caffeic acid, and cinnamic acid. Other suitable acids include, for example, citric acid, lactic acid, oxalic acid, and maleic acid. However, for cholesterol-lowering effects, and other associated health benefits, fatty acid esterification is preferred. For example, mixtures of the fatty acids of any vegetable oil can be used. One example is a mixture of rapeseed oil and rapeseed oil fatty acid methyl ester. The preferred fatty acids useful herein are selected from saturated straight chain fatty acids, saturated branched chain fatty acids, and unsaturated fatty acids. The carbon chain length of the fatty acid useful in the present invention is preferably from 2 to about 24, more preferably from about 12 to about 24, even more preferably from about 16 to about 20, and most preferably about 18.

Suitable examples of fatty acids useful for esterification herein include, for example, valeric acid, isovaleric acid, sorbic acid, isocaproic acid, lauric acid, myristic acid, palmitic acid, stearic acid, arachidic acid, behenic acid, hexacosanoic acid, octacosanoic acid, pentadecanoic acid, heptadecanoic acid nonadecanoic acid, tricosanoic acid, petacosanoic acid, decenylic acid, undecenylic acid, dodecenylic acid, oleic acid, linoleic acid, linolenic acid, arachidonic acid, erucic acid, acetic acid, propionic acid, butyric acid, caproic acid, caprylic acid, and capric acid. More preferred fatty acids include lauric acid, palmitic acid, stearic acid, arachidic acid, behenic acid, oleic acid, cetoleic acid, erucic acid, elaidic acid, linoleic acid, and linolenic acid. Additionally, fatty acid mixtures may be utilized, for example, mixtures of rice bran oil, sunflower oil, safflower oil, rapeseed oil, linseed oil, linola oil, and/or soybean oil may be utilized.

Such fatty acids may be utilized to provide the sterol and/or stanol fatty acid ester. Again, any of the foregoing sterols and stanols may be utilized, with the preferred limitations for sterols and stanols (e.g., phytosterols and stanols derived therefrom) being applicable for the fatty acid esters as well. For example, sitostanol fatty acid esters (e.g., □-sitostanol fatty acid esters) are particularly preferred for use herein. Non-limiting examples of fatty acid esters include sitosterol acetate, sitosterol oleate, and stigmasterol oleate. Of course, the corresponding sitostanol fatty acid esters may also be utilized, e.g., sitostanol acetate, sitostanol oleate, and sitgmastanol oleate.

Other non-limiting examples of fatty acid sterol and stanol esters include ergosta-5,7-dien-3-ol-9-hexadecenoate; (ergosta-5,7-dienylpalmitoleate); ergosta-8,22-dien-3-ol-14-methyl-4,9-octadecenoate; (14-□-methylergosta-8,22-dienyloleate); lanost-8-en-3-ol-9-octadecenoate; (dihydrolanosterol-oleate); ergost-5-en-3-ol-9,12,15-octadecatrienoate; dihydrobrassicasteryl-linolenate; ergost-5-en-3-ol-9,12-octadecadienoate; ergost-5-en-3-ol-9-octadecenoate; dihydrobrassicasteryl-oleate; ergosta-7,24 (28)-dien-3-ol-4-methyl-9-octadecenoate; gramisteryl-oleate; stigmasta-8,24 (28)-dien-3-ol-9,12-octadecadienoate; □⁷-avenasteryl-linoleate; ergosta-7,24 (28)-dien-3-ol-4-methyl-9,12-octadecadienoate; gramisteryl-linoleate; stigmast-24 (28)-en-3-ol-9,12-octadecadienoate; ergosta-5,22-dien-3-ol-4,23-dimethyl-9-octadecenoate; ergostan-3-ol-4-methyl-9-octadecenoate; 5□-stigmastan-3□-ol-linolenate; 5□-stigmastan-3-ol-oleate; stigmastan-3-ol-9,12-octadecadienoate; 5□-stigmastan-3□-ol-linoleate; 22-dihydrospinasteryl-linoleate; ergosta-5,7,22-trien-3-ol-9,12-octa-decadienoate; ergosterol-linoleate; stigmasta-5,24 (28)-dien-3-ol-9-octadecenoate; stigmasta-5,24 (28)-3-ol-9,12-octadecadienoate; stigmasta-5-en-3-ol-5,8,11,14-eicosatetraenoate; □-sitosterol-arachidonate; ergost-5-en-3-ol-5,8,11,14-eicosatetraenoate; stigmasta-7,24 (28)-dien-3-ol-4-methyl-9,12-octadecadienoate; ergost-7-en-3-ol-9,12,15-octadecatrienoate; ergost-5-en-3-ol-9,12,15-octadecatrienoate; campesteryl-linolenate; ergostan-3-ol-9,12-octadecadienoate; ergosta 5,24 (28)-dien-3-ol-9-hexadecenoate; ergosta-5,22-dien-3-ol-octadecenoate; brassicasteryl-oleate; lathosteryl-oleate; lanosta-8,24-dien-3-ol-9-octadecenoate; lanosterol-oleate; stigmasta-5,24(28)-dien-3-ol-9-octadecenoate; fucosteryl-oleate; desmosteryl-oleate; ergost-5-en-3-ol-12-octadecadienoate; campesteryl-linoleate; ergosta-5,22-dien-3-ol-9-octadecenoate; ergost-22-en-3-ol-9-hexadecenoate; ergosta-5,22-dien-3-ol-9-hexadecenoate; ergosta-5,22-dien-3-ol-9,12-octadecadienoate; brassicasteryl-linoleate; ergosta-7,24(28)-dien-3-ol-9,12-octadecadienoate; stigmasta-5,22-dien-3-ol -9,12,15-octadecatrienoate; stigmasterol-linolenate; stigmasta-5,22-dien -3-ol-9,12-octadecadienoate; stigmasterol-linoleate; zymosteryl-oleate; ergost-5-en-3-ol -9-octadecenoate; campesteryl-oleate; ergosta-5,7,22-trien-3-ol-9-hexadecenoate; ergosterol-9-hexadecenoate; 5□-stigmasta-7,22-dien-3□-ol-oleate; □-spinasterol-oleate; ergosta-5,7,22-trien-3-ol-9-octadecenoate; ergosterol-oleate; stigmast-5-en-3-ol -9-octadecenoate; □-sitosterol-oleate; stigmast-5-en-3-ol-9,12-octadecadienoate; □-sitosterol-linoleate; stigmast-5-en-3-ol-9,12,15-octadecatrienoate; □-sitosterol-linolenate; □-sitosterol-undecenoate; □-sitosterol-lauroylate; □-sitosterol-palmitate; stigmasterol-undecenoate; stigmasterol-lauroylate; stigmasterol-palmitate; □-sitostanol-oleate; □-sitostanol-linoleate; □-sitostanol-linolenate; □-sitosterol-oleate; 5□-stigmastan-3□-ol-oleate; 5□-stigmastan-3□-ol-linolenate; 10□-ergosta-5,7,22-trien-3□-ol-linoleate; stigmast-5-en 3-ol-dodecenoate; □-sitosterol-dodecenoate); ergost-5-en-3-ol-dodecenoate; campesteryl-dodecenoate; stigmasterol-dodecenoate; and □-sitosterol-dodecenoate.

Preparation of such fatty acid sterols and/or stanols are well-known to one of ordinary skill in the art. For example, Van Amerongen et al., WO 98/01126, assigned to Unilever PLC, published Jan. 15, 1998, describes processes for the manufacture of a mixture of fatty acid esters comprising hydrolyzing a sterol ester or a mixture of sterol esters and esterifying the so obtained free sterols with particular fatty acids. Preferably, the conditions of the esterification reaction are chosen such that at least 50 wt %, preferably at least 75 wt %, and most preferably from 90-100 wt % of the sterols and/or stanols are esterified. Other methods are disclosed in various references, for example, Miettenen et al., U.S. Pat. No. 5,958,913, assigned to Raisio Benecol Ltd., issued Sep. 28, 1999, which briefly describes esterification at a temperature of 90° C. to 120° C. under a vacuum of 5 to 15 mm Hg and using a catalyst such as sodium ethylate.

Polyol Fatty Acid Polyesters

As an alternative to the sterols, stanols, and/or esters thereof, the second component may be selected from polyol fatty acid polyesters. Polyol fatty acid polyesters, and methods of their synthesis, are commonly known to provide no-fat or reduced calorie foods. Such polyol fatty acid polyesters are disclosed in, for example, Fulcher, U.S. Pat. No. 4,582,927, issued Apr. 15, 1986 (fatty esters of malonic acid), Volpenhein, U.S. Pat. No. 4,582,715, issued Apr. 15, 1986 (□-acetylated triglycerides), Whyte, U.S. Pat. No. 3,579,548, and issued May 18, 1991 (triglycerides of □-branched chain carboxylic acids). Other references which describe useful polyol fatty acid polyesters include Letton et al., U.S. Pat. No. 5,306,514, issued Apr. 26, 1994; Letton et al., U.S. Pat. No. 5,306,515, issued Apr. 26, 1994; Johnston et al., U.S. Pat. No. 5,451,416, issued Sep. 19, 1995; and Elsen et al., U.S. Pat. No. 5,422,131, issued Jun. 6, 1995. Polyol fatty acid polyesters may also be utilized in combination with, e.g., triglycerides, to provide low-fat foods. For example, Seiden et al., U.S. Pat. No. 5,419,925, issued May 30, 1995 describes reduced calorie fat compositions which contain combinations of polyol fatty acid polyesters and certain reduced calorie triglycerides.

The present inventors have surprisingly discovered that the first component, as described herein, may be combined with a polyol fatty acid polyester to provide several unexpected benefits. For example, it is known that certain polyol fatty acid polyesters are sensitive to oxidation and may oxidatively decompose under certain conditions. However, the present inventors have discovered that inclusion of the first component reduces susceptibility of the polyol fatty acid polyester to oxidative decomposition. As an additional unexpected benefit, the polyol fatty acid polyester diminishes and/or removes the undesirable flavor typically associated with the first component of the composition. Accordingly, as discovered herein, use of a polyol fatty acid polyester as the second component provides several unexpected advantages which have not been previously recognized.

Preferred among the polyol fatty acid polyesters are sucrose polyesters (i.e., sucrose in which at least four of the eight hydroxyl groups are esterified with a fatty acid). Sucrose polyester is a nondigestible fat which as been utilized in a variety of food compositions to provide non-fat foods. Such sucrose polyesters are described in, for example, the foregoing references. Particularly preferred sucrose polyesters are those sold under the trade name OLEAN™ and/or OLESTRA™, by Procter & Gamble Co., Cincinnati, Ohio.

Flowable non-digestable polyol fatty acid polyesters, including sucrose polyesters, are also particularly preferred herein. Flowable non-digestable polyol fatty acid polyesters and processes for making such polyol fatty acid polyesters are disclosed in Cerreta et al., U.S. patent application Ser. No. 08/844,590, filed Apr. 21, 1997.

### Kits of the Present Invention

The present invention further relates to kits comprising a composition as described herein and information that use of the composition provides treatment against general health benefits. Such general health benefits include, but are not limited to, cardiovascular benefits, including lowering cholesterol in the consumer, treating, preventing, and/or inhibiting heart disease (e.g., atherosclerosis, restenosis, thrombosis) and, for example, treating other conditions such as hypercholesterolemia, hypertension, poor circulation, and other complications associated with diabetes. Additionally, the kit may comprise information that use of the compound/composition provides an organoleptic benefit, for example acceptable (e.g., good) flavor.

The information provided within the kit may for example, be oral information disseminated as part of the kit, but is preferably written information. Such written information is typically present on packaging associated with the composition (e.g., a label present on a package containing the composition or package insert included within the kit). As used herein, “written” means through words, pictures, symbols, and/or other visible information. Such information need not utilize the actual words but rather use of pictures, symbols, and the like conveying the same or similar meaning are contemplated within the scope of this invention. Such information may also include information about general health benefits and reasons for which such health, and particularly treatment against certain disease states (including the aforementioned disease states), is important for the user.

### Methods of the Present Invention

The present invention also encompasses methods for providing certain health benefits, particularly, lowering serum cholesterol or treating other cardiovascular problems or diseases (as set forth herein) comprising systemically (generally, orally) administering to a mammal (preferably, a human) successive therapeutically effective doses of the present compositions. Such methods include treating, preventing, and/or inhibiting (collectively referred to herein as treating) one or more of the following: cardiovascular problems including, but not limited to, atherosclerosis, restenosis, thrombosis, hypercholesterolemia, hypertension, diabetes, vascular dysfunction, and poor circulation, and other problems such as shock. Preferred methods herein include treatment of one or more of atherosclerosis, hypercholesterolemia, hypertension, diabetes, and poor circulation.

In accordance with the methods of the present invention, a present composition is administered to a mammal, preferably a human. Preferably such administration is oral. As used herein, the term “oral administration” (or the like) with respect to the mammal (preferably, human) means that the mammal ingests or is directed to ingest (preferably, for the purpose of treatment of one or more of the various health problems described herein) one or more compositions of the present invention. Wherein the mammal is directed to ingest one or more of the compositions, such direction may be that which instructs and/or informs the user that use of the composition may and/or will provide treatment for the particular health problem of concern. For example, such direction may be oral direction (e.g., through oral instruction from, for example, a physician, sales professional or organization, and/or radio or television media (i.e., advertisement) or written direction (e.g., through written direction from, for example, a physician or other medical professional (e.g., scripts), sales professional or organization (e.g., through, for example, marketing brochures, pamphlets, or other instructive paraphernalia), written media (e.g., internet, electronic mail, or other computer-related media), and/or packaging associated with the composition (e.g., a label present on a package containing the composition). As used herein, “written” means through words, pictures, symbols, and/or other visible descriptors.

Administration of the present compositions may be via any systemic method, however, such administration is preferably oral. Typically such administration is at least once monthly, but preferably weekly, and most preferably daily. Preferred dosages of the present compositions will vary. As one of ordinary skill will recognize such variations are largely dependent upon factors such as age, gender, weight, and health state of the consumer. However, it is often preferred that from about 0.05 grams to about 50 grams of the first component is administered daily in such composition. More preferably, from about 0.01 grams to about 20 grams, even more preferably from about 0.1 gram to about 10 grams, and most preferably from about 0.5 grams to about 6 grams of the first component is administered daily in such composition. Additionally, as a daily dose, typically from about 0.01 grams to about 50 grams of the second component is administered daily. More preferably, from about 0.05 grams to about 20 grams, even more preferably from about 0.1 gram to about 6 grams, and most preferably from about 0.2 grams to about 4 grams of the second component is administered daily.

### Method of Making the Present Compositions

In accordance with the present invention, the mixture of the first component and the second component results in a thorough coating of the first component (L-arginine, salt, peptide, or pro-form thereof) which, as has been surprisingly discovered herein, diminishes or removes the strong, bitter, and/or fishy flavor characteristics of L-arginine. Several methods, including simple mixture of the first and second components will be well-known in the art. However, for convenience, the following is a non-limiting example of a method of making the present compositions.

The particle size of the L-arginine (or salt, polypeptide, or pro-form thereof) should be reduced to minimize the perception of arginine particles in the mouth, when consuming the finished product. Typically such particle size will be less than about 100 microns. The coating material is a second component of the present invention, as has been described herein. The coating material may be mixed with one or more triglycerides, such as □-3 fatty acids, to make the coating materials more plastic or deformable, which in turn provides enhanced texture of the final product.

The solvent system utilized is preferably an azeotropic mixture such that during the drying process solvent ratios are relatively maintained; such maintenance will enhance the solubility of the coated material. It has further been found that using the azeotropic mixture of solvents results in a uniform smooth thin film of coated material. Additionally, it has been discovered that wherein the azeotropic solvent system is not used the coated material can precipitate. Such precipitate will typically cause the coating to be lumpy and granular and (resulting in multiple particles sticking together) rather than smooth and uniform. It is important to avoid such precipitate because the precipitate can cause enhanced perceptibility of unacceptable flavor. Actual coating of the first component herein by the second component herein is described in the following non-limiting examples. Variations of the following will be well-known to one of ordinary skill with the benefit of the present disclosure.

### EXAMPLE 1

A composition is prepared as follows having 40% phytosterol and 60% L-arginine. Phytosterols (333 grams, commerically available from ADM, Decatur, Ill.) are coated onto L-arginine using a solvent system of hexane and ethanol. A mixture of 17% phytosterol, 69% hexane, and 21% ethanol is heated to 55° C. L-arginine (about 500 grams) is loaded into a lab model Lakso Wurster Coater having a 4-inch by 6-inch bowl (Model 101, commercially available from Lakso Co., Leominster, Mass.). A ¼ J-type two-fluid nozzle having a fluid capacity of 20/50, air capacity of 70 manufactured by Spraying Systems Co., Wheaton, Ill., is used to spray the phytosterol mixture. The gap between the Wurster insert and the distributor plate is adjusted depending upon the particle size of the L-arginine. A peristaltic pump is used to pump the phytosterol mixture to the nozzle. The mixture is circulated through the pumping system. The phytosterol solution should be adequately mixed by using a stir bar. The air flow to the unit is started. The air flow is adjusted until the bed is fluidizing correctly. The humidity of the inlet air to the bed is adjusted if necessary. The inlet air temperature is adjusted between about 100° C. and about 140° C. The mixture is fluidized at the desired air flow. The blowback to the filter bags is turned on. The air pressure to the nozzle is adjusted to between about 20 and about 24 psi. When the desired bed temperature is achieved, the flow of the phytosterol mixture to the nozzle block is started. When all of the phytosterol mixture is sprayed, the bed is dried under ambient temperature and pressure for about 10 minutes before turning off to allow the resulting composition to harden. After all appropriate settings are turned off, the bowl is unclamped and the phytosterol-coated L-arginine is removed.

### EXAMPLE 2

Example 1 is repeated with use of 17% phytosterol and 83% L-arginine to provide a composition providing substantially similar results.

### EXAMPLE 3

Example 1 is repeated with use of 60% phytosterol and 40% L-arginine to provide a composition providing substantially similar results.

### EXAMPLE 4

Example 1 is repeated with use of 23% phytosterol and 77% L-arginine to provide a composition providing substantially similar results. The coating material is a mixture of 33% stigmasterol and 67% sitosterols, both supplied by Sigma Chemical Co., St. Louis, Mo., in solvent system. The coating material contains 15% sterol and 85% solvent system.

### EXAMPLE 5

Example 1 is repeated with use of 35% stigmasterol (supplied by Sigma Chemical Co., St. Louis, Mo.) and 65% L-arginine to provide a composition providing substantially similar results.

### Use of the Present Compositions and Kits

The compounds described herein can be used in compositions comprising fat and non-fat components to provide general health benefits, including cardiovascular benefits, such as lowering cholesterol in the consumer, treating, preventing, and/or inhibiting heart disease (e.g., atherosclerosis, restenosis, thrombosis) and, for example, treating other conditions such as hypertension, poor circulation, and complications associated with diabetes. The compositions are useful in a wide variety of finished products, including pharmaceutical, food, and beverage products.

Preferred herein is use of the present compositions in food products, including those envisioned for use as a dietary supplement such as a health bar. In a preferred embodiment of the present invention, the compositions is in the form of a health bar.

As non-limiting examples, the compounds can be used in the production of baked goods in any form, such as mixes, shelf-stable baked goods (including health bars), and frozen baked goods. Applications include, but are not limited to, cakes, brownies, muffins, bar cookies, health bars, wafers, biscuits, pastries, pies, pie crusts, and cookies, including sandwich cookies and chocolate chip cookies, particularly the storage-stable dual-textured cookies described in Hong et al., U.S. Pat. No. 4,455,333. The baked goods can contain fruit, cream, or other fillings. Other baked good uses include breads and rolls, crackers, pretzels, pancakes, waffles, ice cream cones and cups, yeast-raised baked goods, pizzas and pizza crusts, baked farinaceous snack foods, and other baked salted snacks.

As stated, health bars are a particularly preferred embodiment of the present invention. The compounds can be incorporated into health bars, such as those described in Greenberg et al., U.S. Pat. No. 5,780,039. The foregoing doses of the present compounds may be included in the advantageous health bars according to the present invention.

In addition to their uses in baked goods, the compositions herein can be used alone or in combination with fats to make shortening and oil products. The fats can be synthetic or derived from animal or vegetable sources, or combinations of these. Shortening and oil products include, but are not limited to, shortenings, margarines, spreads, butter blends, lards, cooking and frying oils, salad oils, popcorn oils, salad dressings, mayonnaise, and other edible oil products. In a particular embodiment of the present invention, the compositions are selected from margarines, butter, dressings and spreads.

Other uses for the compositions of the present invention include partial or complete replacement fats and/or oils present in peanut butter, frozen desserts such as ice cream and ice cream coatings, whipped toppings, frosting products, processed meat products, including vegetable protein-based meat analog products, sauces, gravies, and dairy products such as milkshakes, milk products, coffee whiteners, and cheese products.

The compounds described herein are also particularly useful in beverage compositions. Such beverage compositions may be dilute water beverages (also called “near-water” beverages), milks, coffees, teas, colas, and fruit juices.

The compositions of the present invention may comprise one or more of the following optional ingredients:

The isothiocyanate compound as described herein (optionally together with the sorbate or benzoate preservative) is particularly useful in beverage products, especially dilute juice beverages, fortified beverages (e.g., calcium fortified beverage), beverage products containing tea solids (i.e., teas), and beverages containing milk solids. The isothiocyanate compound is most preferably present in the aqueous phase of the beverage product for effective antimicrobial effect. Preferred beverage products of the present invention are those comprising a beverage member selected from the group consisting of water, fruit juice, tea solids, milk solids, fruit flavors, botanical flavors, and mixtures thereof. The beverage products herein are most preferably dilute juice beverages (particularly fruit juice beverages) and beverages containing tea solids, and beverage products comprising fruit juice and tea solids. Particularly preferred beverage products comprise both fruit juice and water. Other particularly preferred beverage products comprise both tea solids and water. In another preferred embodiment, “near water” (lightly flavored water) is utilized.

Various optional elements may be incorporated into the products and methods of the present invention. Non-limiting examples of optional elements are as follows:

Water

Water may be included in the compositions of the present invention, particularly wherein the compositions are beverage compositions. As used herein, the term “water” includes the total amount of water present in the composition. “Water” includes water from flavor agents, sugar syrups, and other sources, e.g., gum solutions. Water of hydration of, for example, calcium and other solids, is also included. Wherein water is included, water is preferably included at levels from about 0.1% to about 99.999%, more preferably from about 5% to about 99%, still more preferably from about 40% to about 95%, even more preferably from about 50% to about 90%, and most preferably from about 70% to about 90%, by weight of the composition.

Beverage Emulsions

Dilute juice beverages of the present invention may optionally, but preferably, comprise from about 0.2% to about 5%, preferably from about 0.5% to about 3%, and most preferably from about 0.8% to about 2%, of a beverage emulsion. This beverage emulsion can be either a cloud emulsion or a flavor emulsion.

For cloud emulsions, the clouding agent can comprise one or more fats or oils stabilized as an oil-in-water emulsion using a suitable food grade emulsifier. Any of a variety of fats or oils may be employed as the clouding agent, provided that the fat or oil is suitable for use in foods and/or beverages. Preferred are those fats and oils that have been refined, bleached and deodorized to remove off-flavors. Especially suitable for use as clouding agents are those fats that are organoleptically neutral. These include fats from the following sources: vegetable fats such as soybean, corn, safflower, sunflower, cottonseed, canola, and rapeseed; nut fats such as coconut, palm, and palm kernel; and synthetic fats. See e.g., Kupper et al., U.S. Pat. No. 4,705,691, issued Nov. 10, 1987, for suitable fat or oil clouding agents.

Any suitable food grade emulsifier can be used that can stabilize the fat or oil clouding agent as an oil-in-water emulsion. Suitable emulsifiers include gum acacia, modified food starches (e.g., alkenylsuccinate modified food starches), anionic polymers derived from cellulose (e.g., carboxymethylcellulose), gum ghatti, modified gum ghatti, xanthan gum, tragacanth gum, guar gum, locust bean gum, pectin, and mixtures thereof. See e.g., Kupper et al., U.S. Pat. No. 4,705,691, issued Nov. 10, 1987. Modified starches treated to contain hydrophobic as well as hydrophilic groups, such as those described in Caldwell et al., U.S. Pat. No. 2,661,349, are preferred emulsifiers for use as herein. Octenyl succinate (OCS) modified starches such as those described in Marotta et al., U.S. Pat. No. 3,455,838 and Barndt et al., U.S. Pat. No. 4,460,617 are especially preferred emulsifiers.

The clouding agent can be combined with a weighting agent to provide a beverage opacifier that imparts a total or partial opaque effect to the beverage without separating out and rising to the top. The beverage opacifier provides the appearance to the consumer of a juice-containing beverage. Any suitable weighting oil can be employed in the beverage opacifier. Typical weighting oils include brominated vegetable oil, glycerol ester of wood rosin (ester gum), sucrose acetate isobutyrate (SAIB) and other sucrose esters, gum damar, colophony, gum elemi, or others known to those skilled in the art. Other suitable weighting agents include brominated liquid polyol polyesters which are nondigestible. See e.g., Brand et al., U.S. Pat. No. 4,705,690, issued Nov. 10, 1987.

The cloud/opacifier emulsion is prepared by mixing the clouding agent with the weighting agent (for opacifier emulsions), the emulsifier and water. The emulsion typically contains from about 0.1% to about 25% clouding agent, from about 1% to about 20% weighting oil agent (in the case of opacifier emulsions), from about 1% to about 30% emulsifiers, and from about 25% to about 97.9% water (or quantum satis).

The particle size of the water-insoluble components of the emulsion is reduced by employing a suitable apparatus known in the art. Because the ability of emulsifying agents to hold oil in suspension is proportional to particle size, emulsions of particles with diameters of about 0.1 to about 3.0 microns are suitable. Preferably, the particles are about 2.0 microns or less in diameter. Most preferred is an emulsion in which substantially all the particles are 1.0 microns or less in diameter. The particle size is reduced by passing the mixture through an homogenizer, colloid mill or turbine-type agitator. Usually one or two passes is sufficient. See e.g., Kupper et al., U.S. Pat. No. 4,705,691, issued Nov. 10, 1987.

Flavor emulsions useful in beverage products of the present invention comprise one or more suitable flavor oils, extracts, oleoresins, essential oils and the like, known in the art for use as flavorants in beverages. This component can also comprise flavor concentrates such as those derived from concentration of natural products such as fruits. Terpeneless citrus oils and essences can also be used herein. Examples of suitable flavors include, for example, fruit flavors such as orange, lemon, lime and the like, cola flavors, tea flavors, coffee flavors, chocolate flavors, dairy flavors. These flavors can be derived from natural sources such as essential oils and extracts, or can be synthetically prepared. The flavor emulsion typically comprises a blend of various flavors and can be employed in the form of an emulsion, alcoholic extract, or spray dried. The flavor emulsion can also include clouding agents, with or without weighting agents, as previously described. See e.g., Kupper et al., U.S. Pat. No. 4,705,691, issued Nov. 10, 1987.

Flavor emulsions are typically prepared in the same manner as cloud/opacifier emulsions by mixing one or more flavoring oils (from about 0.001% to about 20%) with an emulsifying agent (from about 1% to about 30%) and water. (The oil clouding agents can also be present). Emulsions of particles with diameters of from about 0.1 to about 3.0 microns are suitable. Preferably, the particles are about 2.0 microns or less in diameter. Most preferably, the particles are about 1.0 microns or less in diameter. The emulsifying agent coats the particularized flavor oil to aid in preventing coalescence and in maintaining an appropriate dispersion. The viscosity and specific gravity of the flavor emulsion are regulated to be compatible with the finished beverage. See e.g., Kupper et al., U.S. Pat. No. 4,705,691, issued Nov. 10, 1987.

Flavor Agents

The compositions herein may optionally, but preferably, comprise one or more flavor agents. Preferably, such flavor agents are included in the beverage compositions and are typically selected from fruit juice, tea solids, milk solids, fruit flavors, botanical flavors, and mixtures thereof. Wherein fruit juice is included, the beverages of the present invention can comprise from about 0.1% to about 40%, preferably from about 1% to about 20%, more preferably from about 2% to about 10%, and most preferably from about 3% to about 6%, fruit juice. (As measured herein, the weight percentage of fruit juice is based on a single strength 2° to 16° Brix fruit juice). The fruit juice can be incorporated into the beverage as a puree, comminute, or as a single strength or concentrated juice. Especially preferred is incorporation of the fruit juice as a concentrate with a solids content (primarily as sugar solids) of from about 20° to about 80° Brix.

The fruit juice can be any citrus juice, non-citrus juice, or mixture thereof, which are known for use in dilute juice beverages. The juice can be derived from, for example, apple, cranberry, pear, peach, plum, apricot, nectarine, grape, cherry, currant, raspberry, gooseberry, elderberry, blackberry, blueberry, strawberry, lemon, lime, mandarin, orange, grapefruit, cupuacu, potato, tomato, lettuce, celery, spinach, cabbage, watercress, dandelion, rhubarb, carrot, beet, cucumber, pineapple, coconut, pomegranate, kiwi, mango, papaya, banana, watermelon, passion fruit, tangerine, and cantaloupe. Preferred juices are derived from apple, pear, lemon, lime, mandarin, grapefruit, cranberry, orange, strawberry, tangerine, grape, kiwi, pineapple, passion fruit, mango, guava, raspberry and cherry. Citrus juices, preferably grapefruit, orange, lemon, lime, and mandarin juices, as well as juices derived from mango, apple, passion fruit, and guava, as well as mixtures of these juices are most preferred.

Fruit flavors may also be utilized. As described above with respect to flavor emulsions, fruit flavors may be derived from natural sources such as essential oil and extracts, or can be synthetically prepared. Fruit flavors may be derived from fruits through processing, particularly concentrating. Wherein fruit juices are concentrated or evaporated, the water which is removed or the condensate contains volatile substances which comprise the flavor of the fruit. Often, such flavor is added to a juice concentrate to enhance the flavor thereof. The condensate may also be used to flavor “near waters” (lightly flavored water).

Botanical flavors may also be utilized. As used herein, the term “botanical flavor” refers to a flavor derived from parts of a plant other than the fruit; i.e., derived from nuts, bark, roots, and/or leaves. Also included within the term “botanical flavor” are synthetically prepared flavors made to simulate botanical flavors derived from natural sources. Botanical flavors can be derived from natural sources such as essential oils and extracts, or can be synthetically prepared. Suitable botanical flavors include jamaica, kola, marigold, chrysanthemum, chamomile, ginger, valerian, yohimbe, hops, eriodictyon, ginseng, bilberry, rice, red wine, mango, peony, lemon balm, nut gall, oak chip, lavender, walnut, gentiam, luo han guo, cinnamon, angelica, aloe, agrimony, yarrow and mixtures thereof.

Tannic acid or other similar acids can be used to provide an astringent taste to the beverage. From about 0.001% to about 10% tannic acid is used. Other flavor enhancers, as well as flavorants such as chocolate and vanilla can also be used.

Wherein tea solids are included, the beverages of the present invention can comprise from about 0.01% to about 1.2%, preferably from about 0.05% to about 0.8%, by weight of the beverage product, of tea solids. The term “tea solids” as used herein means solids extracted from tea materials including those materials obtained from the genus Camellia including C. sinensis and C. assaimica, for instance, freshly gathered tea leaves, fresh green tea leaves that are dried immediately after gathering, fresh green tea leaves that have been heat treated before drying to inactivate any enzymes present, unfermented tea, instant green tea, and partially fermented tea leaves. Green tea materials are tea leaves, tea plant stems, and other plant materials that are related and which have not undergone substantial fermentation to create black teas. Members of the genus Phyllanthus, Catechu gambir and Uncaria family of tea plants can also be used. Mixtures of unfermented and partially fermented teas can be used.

Tea solids for use in beverages of the present invention can be obtained by known and conventional tea solid extraction methods. A particularly preferred source of green tea solids can be obtained by the method described in Ekanayake et al., U.S. application Ser. No. 08/606,907, filed Feb. 26, 1996. Tea solids so obtained will typically comprise caffeine, theobromine, proteins, amino acids, minerals and carbohydrates. Suitable beverages containing tea solids can be formulated according to Tsai et al., U.S. Pat. No. 4,946,701, issued Aug. 7, 1990. See also, Ekanayake et al., U.S. Pat. No. 5,427,806, issued Jun. 26, 1995, for a suitable sources of green tea solids for use in the present invention.

Beverages according to the present invention may also comprise milk solids. These milk solids can be derived from various sources including whole milk, skim milk, condensed milk, and dried milk powder. As used herein, the term “milk” will be used to describe an aqueous dispersion of milk solids, such as fluid (whole or skim milk) or non-fat dry milk or condensed milk diluted with water. The amount of milk included typically ranges from about 5% to about 99.8%, preferably from about 5% to about 75%, more preferably from about 5% to about 40%, and most preferably from about 5% to about 15%. The amount of non-fat milk solids correlating to these levels of milk solids is in the range of from about 0.5% to about 8.2%, from about 0.5% to about 6.2%, from about 0.5% to about 3.3%, and from about 0.5% to 1.2% of the beverage, respectively.

Thickeners and Bulking Agents

Food and beverage compositions according to the present invention can further comprise thickeners, including xanthan gum, carboxymethylcellulose, carboxyethylcellulose, hydroxypropylcellulose, methylcellulose, microcrystalline cellulose, starches, dextrins, fermented whey, tofu, maltodextrins, polyols, including sugar alcohols (e.g., sorbitol and mannitol), carbohydrates (e.g., lactose), propylene glycol alginate, gellan gum, guar gum, pectin, tragacanth gum, gum acacia, locust bean gum, gum arabic, gelatin, as well as mixtures of these thickeners. These thickeners are typically included in the compositions of the present invention at levels up to about 0.1%, depending on the particular thickener involved and the viscosity effects desired.

Sweeteners

The food and beverage compositions of the present invention can, and typically will, contain an effective amount of one or more sweeteners, including carbohydrate sweeteners and natural and/or artificial no/low calorie sweeteners. The amount of the sweetener used in the compositions of the present invention typically depends upon the particular sweetener used and the sweetness intensity desired. For no/low calorie sweeteners, this amount varies depending upon the sweetness intensity of the particular sweetener.

The compositions of the present invention can be sweetened with any of the carbohydrate sweeteners, preferably monosaccharides and/or disaccharides. Sweetened compositions, particularly beverages, will typically comprise from about 0.1% to about 20%, most preferably from about 6 to about 14%, sweetener. These sweeteners can be incorporated into the compositions in solid or liquid form but are typically, and preferably, incorporated as a syrup, most preferably as a concentrated syrup such as high fructose corn syrup. For purposes of preparing beverages of the present invention, these sugar sweeteners can be provided to some extent by other components of the beverage such as, for example, the fruit juice component and/or flavors.

Preferred sugar sweeteners for use in compositions of the present invention are sucrose, fructose, glucose, and mixtures thereof. Fructose can be obtained or provided as liquid fructose, high fructose corn syrup, dry fructose or fructose syrup, but is preferably provided as high fructose corn syrup. High fructose corn syrup (HFCS) is commercially available as HFCS-42, HFCS-55 and HFCS-90, which comprise 42%, 55% and 90%, respectively, by weight of the sugar solids therein, as fructose. Other naturally occurring sweeteners or their purified extracts, such as glycyrrhizin, the protein sweetener thaumatin, the juice of Luo Han Guo disclosed in, for example, Fischer et al., U.S. Pat. No. 5,433,965, issued Jul. 18, 1995, and the like can also be used in the compositions of the present invention.

Suitable no/low calorie sweeteners include saccharin, cyclamates, L-aspartyl-L-phenylalanine lower alkyl ester sweeteners (e.g., aspartame); L-aspartyl-D-alanine amides disclosed in Brennan et al., U.S. Pat. No. 4,411,925; L-aspartyl-D-serine amides disclosed in Brennan et al., U.S. Pat. No. 4,399,163; L-aspartyl-L-1-hydroxymethylalkaneamide sweeteners disclosed in Brand, U.S. Pat. No. 4,338,346; L-aspartyl-1-hydroxyethyalkaneamide sweeteners disclosed in Rizzi, U.S. Pat. No. 4,423,029; L-aspartyl-D-phenylglycine ester and amide sweeteners disclosed in Janusz, European Patent Application 168,112, published Jan. 15, 1986; N-[N-3,3-dimethylbutyl)-L-□-aspartyl]-L-phenylalanine 1-methyl ester sweeteners disclosed in Gerlat et al., WO 99/30576, assigned to The Nutrasweet Co., published Jun. 24, 1999; alltame, thaumatin; dihydrochalcones; cyclamates; steviosides; glycyrrhizins, synthetic alkoxy aromatics, such as Dulcin and P-4000; sucrolose; suosan; miraculin; monellin; sorbitol, xylitol; talin; cyclohexylsulfamates; substituted imidazolines; synthetic sulfamic acids such as acesulfame, acesulfame-K and n-substituted sulfamic acids; oximes such as perilartine; rebaudioside-A; peptides such as aspartyl malonates and succanilic acids; dipeptides; amino acid based sweeteners such as gem-diaminoalkanes, meta-aminobenzoic acid, L-aminodicarboxylic acid alkanes, and amides of certain alpha-aminodicarboxylic acids and gem-diamines; and 3-hydroxy-4-alkyloxyphenyl aliphatic carboxylates or heterocyclic aromatic carboxylates; and the like and mixtures thereof. A particularly preferred low calorie sweetener is aspartame.

Coloring Agent

Small amounts of coloring agents may be utilized in the compositions of the present invention. FD&C dyes (e.g., yellow #5, blue #2, red #40) and/or FD&C lakes are preferably used. By adding the lakes to the other powdered ingredients, all the particles, in particular the colored iron compound, are completely and uniformly colored and a uniformly colored composition is attained. Preferred lake dyes which may be used in the present invention are the FDA-approved Lake, such as Lake red #40, yellow #6, blue #1, and the like. Additionally, a mixture of FD&C dyes or a FD&C lake dye in combination with other conventional food and food colorants may be used. Riboflavin and □-carotene may also be used. The exact amount of coloring agent used will vary, depending on the agents used and the intensity desired in the finished product. The amount can be readily determined by one skilled in the art. Generally, if utilized, the coloring agent should be present at a level of from about 0.0001% to about 0.5%, preferably from about 0.001% to about 0.1%, and most preferably from about 0.004% to about 0.1%, by weight of the composition.

Nutrients

The compositions herein (particularly the food and beverage compositions) can be fortified with one or more nutrients, especially one or more vitamins, minerals, and/or amino acids. The U.S. Recommended Daily Intake (USRDI) for vitamins and minerals are defined and set forth in the Recommended Daily Dietary Allowance-Food and Nutrition Board, National Academy of Sciences-National Research Council.

Any amino acid may be utilized herein, especially the naturally occurring amino acids. Preferred amino acids for inclusion herein are L-lysine and L-carnitine, particularly L-lysine.

Unless otherwise specified herein, wherein a given mineral is present in the product, the product comprises at least about 1%, preferably at least about 5%, more preferably from about 10% to about 200%, even more preferably from about 40% to about 150%, and most preferably from about 60% to about 125% of the USRDI of such mineral. Unless otherwise specified herein, wherein a given vitamin is present in the product, the product comprises at least about 1%, preferably at least about 5%, more preferably from about 10% to about 200%, even more preferably from about 20% to about 150%, and most preferably from about 25% to about 120% of the USRDI of such vitamin.

Non-limiting examples of such vitamins and minerals include iron, zinc, copper, calcium, phosphorous, niacin, thiamin, folic acid, pantothenic acid, iodine, vitamin A, vitamin C, vitamin B₂, vitamin B₃, vitamin B₆, vitamin B₁₂, vitamin D, vitamin E, and vitamin K. Preferably, wherein a vitamin or mineral is utilized the vitamin or mineral is selected from iron, zinc, calcium, niacin, thiamin, folic acid, iodine, vitamin A, vitamin C, vitamin B₆, vitamin B₁₂, vitamin D, and vitamin E. A particularly preferred mineral for use herein is calcium.

Commercially available vitamin A sources may also be included in the present compositions. Vitamin A can be provided, for example, as vitamin A palmitate (retinol palmitate) and/or as beta-carotene. The vitamin A may be in the form of, for example, an oil, beadlets or encapsulated. As used herein, “vitamin A” includes, but is not limited to, vitamin A, β-carotene, retinol palmitate, and retinol acetate. Wherein vitamin A is present in the compositions herein, the product comprises at least about 1%, preferably at least about 5%, more preferably from about 10% to about 200%, even more preferably from about 15% to about 150%, and most preferably from about 20% to about 120% of the USRDI of such vitamin. Wherein vitamin A is present in the products herein, it is especially preferred to include about 25% of the USRDI of vitamin A. The quantity of vitamin A to be added is dependent on processing conditions and the amount of vitamin A deliver desired after storage. Preferably, wherein vitamin A is included within the present compositions, the products comprise from about 0.0001% to about 0.2%, more preferably from about 0.0002% to about 0.12%, also preferably from about 0.0003% to about 0.1%, even more preferably from about 0.0005% to about 0.08%, and most preferably from about 0.001% to about 0.06% of vitamin A, by weight of the composition.

Commercially available sources of vitamin B₂ (also known as riboflavin) may be utilized in the present compositions. Wherein vitamin B₂ is present in the compositions herein, the product comprises at least about 1%, preferably at least about 5%, more preferably from about 5% to about 200%, even more preferably from about 10% to about 150%, and most preferably from about 10% to about 120% of the USRDI of such vitamin. Wherein vitamin B₂ is present in the compositions herein, it is especially preferred to include from about 15% to about 35% of the USRDI of vitamin B₂.

Commercially available sources of vitamin C can be used herein. Encapsulated ascorbic acid and edible salts of ascorbic acid can also be used. Wherein vitamin C is present in the products herein, the product comprises at least about 1%, preferably at least about 5%, more preferably from about 10% to about 200%, even more preferably from about 20% to about 150%, and most preferably from about 25% to about 120% of the USRDI of such vitamin. Wherein vitamin C is present in the compositions herein, it is especially preferred to include about 100% of the USRDI of vitamin C. The quantity of vitamin C to be added is dependent on processing conditions and the amount of vitamin C deliver desired after storage. Preferably, wherein vitamin C is included within the present compositions, the compositions comprise from about 0.005% to about 0.2%, more preferably from about 0.01% to about 0.12%, also preferably from about 0.02% to about 0.1%, even more preferably from about 0.02% to about 0.08%, and most preferably from about 0.03% to about 0.06% of vitamin C, by weight of the composition.

Commercial sources of iodine, preferably as an encapsulated iodine may be utilized herein. Other sources of iodine include iodine-containing salts, e.g., sodium iodide, potassium iodide, potassium iodate, sodium iodate, or mixtures thereof. These salts may be encapsulated.

Nutritionally supplemental amounts of other vitamins which may be incorporated herein include, but are not limited to, vitamins B₆ and B₁₂, folic acid, niacin, pantothenic acid, folic acid, vitamin D, and vitamin E. Wherein the composition comprises one of these vitamins, the product preferably comprises at least 5%, preferably at least 25%, and most preferably at least 35% of the USRDI for such vitamin.

Minerals which may optionally be included in the composition herein are, for example, magnesium, zinc, iodine, iron, and copper. Any soluble salt of these minerals suitable for inclusion edible products can be used, for example, magnesium citrate, magnesium gluconate, magnesium sulfate, zinc chloride, zinc sulfate, potassium iodide, copper sulfate, copper gluconate, and copper citrate.

Calcium is a particularly preferred mineral for use in the present invention. Preferred sources of calcium include, for example, amino acid chelated calcium, calcium carbonate, calcium oxide, calcium hydroxide, calcium sulfate, calcium chloride, calcium phosphate, calcium hydrogen phosphate, calcium dihydrogen phosphate, calcium citrate, calcium malate, calcium titrate, calcium gluconate, calcium realate, calcium tantrate, and calcium lactate, and in particular calcium citrate-malate. The form of calcium citrate-malate is described in, e.g., Mehansho et al., U.S. Pat. No. 5,670,344, issued Sep. 23, 1997; Diehl et al., U.S. Pat. No. 5,612,026, issued Mar. 18, 1997; Andon et al., U.S. Pat. No. 5,571,441, issued Nov. 5, 1996; Meyer et al., U.S. Pat. No. 5,474,793, issued Dec. 12, 1995; Andon et al., U.S. Pat. No. 5,468,506, issued Nov. 21, 1995; Burkes et al., U.S. Pat. No. 5,445,837, issued Aug. 29, 1995; Dake et al., U.S. Pat. No. 5,424,082, issued Jun. 13, 1995; Burkes et al., U.S. Pat. No. 5,422,128, issued Jun. 6, 1995; Burkes et al., U.S. Pat. No. 5,401,524, issued Mar. 28, 1995; Zuniga et al., U.S. Pat. No. 5,389,387, issued Feb. 14, 1995; Jacobs, U.S. Pat. No. 5,314,919, issued May 24, 1994; Saltman et al., U.S. Pat. No. 5,232,709, issued Aug. 3, 1993; Camden et al., U.S. Pat. No. 5,225,221, issued Jul. 6, 1993; Fox et al., U.S. Pat. No. 5,215,769, issued Jun. 1, 1993; Fox et al., U.S. Pat. No. 5,186,965, issued Feb. 16, 1993; Saltman et al., U.S. Pat. No. 5,151,274, issued Sep. 29, 1992; Kochanowski, U.S. Pat. No. 5,128,374, issued Jul. 7, 1992; Mehansho et al., U.S. Pat. No. 5,118,513, issued Jun. 2, 1992; Andon et al., U.S. Pat. No. 5,108,761, issued Apr. 28, 1992; Mehansho et al., U.S. Pat. No. 4,994,283, issued Feb. 19, 1991; Nakel et al., U.S. Pat. No. 4,786,510, issued Nov. 22, 1988; and Nakel et al., U.S. Pat. No. 4,737,375, issued Apr. 12, 1988. Preferred compositions of the present invention will comprise from about 0.01% to about 0.5%, more preferably from about 0.03% to about 0.2%, even more preferably from about 0.05% to about 0.15%, and most preferably from about 0.1% to about 0.15% of calcium, by weight of the composition.

Iron may also be utilized in the compositions of the present invention. Acceptable forms of iron are well-known in the art. The amount of iron compound incorporated into the composition will vary widely depending upon the level of supplementation desired in the final product and the targeted consumer. Iron fortified compositions of the present invention typically contain from about 5% to about 100%, preferably from about 15% to about 50%, and most preferably about 20% to about 40% of the USRDI for iron.

Ferrous iron is typically better utilized by the body than ferric iron. Highly bioavailable ferrous salts that can be used in the ingestible compositions of the present invention are ferrous sulfate, ferrous fumarate, ferrous succinate, ferrous gluconate, ferrous lactate, ferrous tartarate, ferrous citrate, ferrous amino acid chelates, as well as mixtures of these ferrous salts. While ferrous iron is typically more bioavailable, certain ferric salts can also provide highly bioavailable sources of iron. Highly bioavailable ferric salts that can be used in the food or beverage compositions of the present invention are ferric saccharate, ferric ammonium citrate, ferric citrate, ferric sulfate, as well as mixtures of these ferric salts. Combinations or mixtures of highly bioavailable ferrous and ferric salts can be used in these edible mixes and ready-to-serve beverages. The preferred sources of highly bioavailable iron are ferrous fumarate and ferrous amino acid chelates.

Ferrous amino acid chelates particularly suitable as highly bioavailable iron sources for use in the present invention are those having a ligand to metal ratio of at least 2:1. For example, suitable ferrous amino acid chelates having a ligand to metal mole ratio of two are those of formula:

Fe(L)₂

where L is an alpha amino acid, dipeptide, tripeptide, or quadrapeptide ligand. Thus, L can be any ligand which is a naturally occurring alpha amino acid selected from alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine; or dipeptides, tripeptides, or quadrapeptides formed by any combination of these alpha amino acids. See e.g., Ashmead et al., U.S. Pat. No. 4,863,898, issued Sep. 5, 1989; Ashmead, U.S. Pat. No. 4,830,716, issued May 16, 1989; and Ashmead, U.S. Pat. No. 4,599,152, issued Jul. 8, 1986, all of which are incorporated by reference. Particularly preferred ferrous amino acid chelates are those where the reacting ligands are glycine, lysine, and leucine. Most preferred is the ferrous amino acid chelate sold under the mark Ferrochel® (Albion Laboratories, Salt Lake City, Utah) wherein the ligand is glycine.

In addition to these highly bioavailable ferrous and ferric salts, other sources of bioavailable iron can be included in the food and beverage compositions of the present invention. Other sources of iron particularly suitable for fortifying products of the present invention included certain iron-sugar-carboxylate complexes. In these iron-sugar-carboxylate complexes, the carboxylate provides the counterion for the ferrous (preferred) or ferric iron. The overall synthesis of these iron-sugar-carboxylate complexes involves the formation of a calcium-sugar moiety in aqueous media (for example, by reacting calcium hydroxide with a sugar, reacting the iron source (such as ferrous ammonium sulfate) with the calcium-sugar moiety in aqueous media to provide an iron-sugar moiety, and neutralizing the reaction system with a carboxylic acid (the “carboxylate counterion”) to provide the desired iron-sugar-carboxylate complex. Sugars that can be used to prepare the calcium-sugar moiety include any of the ingestible saccharidic materials, and mixtures thereof, such as glucose, sucrose and fructose, mannose, galactose, lactose, maltose, and the like, with sucrose and fructose being the more preferred. The carboxylic acid providing the “carboxylate counterion” can be any ingestible carboxylic acid such as citric acid, malic acid tartaric acid, lactic acid, succinic acid, propionic acid, etc., as well as mixtures of these acids.

These iron-sugar-carboxylate complexes can be prepared in the manner described in, e.g., Nakel et al., U.S. Pat. Nos. 4,786,510 and 4,786,518, issued Nov. 22, 1988, both of which are incorporated by reference. These materials are referred to as “complexes”, but they may exist in solution as complicated, highly hydrated, protected colloids; the term “complex” is used for the purpose of simplicity.

Zinc may also be utilized in the compositions of the present invention. Acceptable forms of zinc are well-known in the art. Zinc fortified products of the present invention typically contain from about 5% to about 100%, preferably from about 15% to about 50%, and most preferably about 25% to about 45% of the USRDI for zinc. The zinc compounds which can be used in the present invention can be in any of the commonly used forms such as, e.g., zinc sulfate, zinc chloride, zinc acetate, zinc gluconate, zinc ascorbate, zinc citrate, zinc aspartate, zinc picolinate, amino acid chelated zinc, and zinc oxide. Zinc gluconate and amino acid chelated zinc are particularly preferred.

Carbonation Component

Carbon dioxide can be introduced into the water which is mixed with a beverage syrup or into the dilute beverage after dilution to achieve carbonation. The carbonated beverage can be placed into a container, such as a bottle or can, and then sealed. Any conventional carbonation methodology may be utilized to make carbonated beverage products of this invention. The amount of carbon dioxide introduced into the beverage will depend upon the particular flavor system utilized and the amount of carbonation desired.

pH

The compositions of the present invention, particularly the beverage compositions, preferably have a pH of from about 2 to about 8, more preferably from about 2 to about 4.5, and most preferably from about 2.7 to about 4.2. Beverage acidity can be adjusted to and maintained within the requisite range by known and conventional methods, e.g., the use of food grade acid buffers. Typically, beverage acidity within the above recited ranges is a balance between maximum acidity for microbial inhibition and optimum acidity for the desired beverage flavor.

Non-Caloric or Reduced Calorie Fats

The compositions can be used in combination with non-caloric or reduced calorie fats, such as branched chain fatty acid triglycerides, triglycerol ethers, polycarboxylic acid esters, sucrose polyesters, sucrose polyethers, neopentyl alcohol esters, silicone oils/siloxanes, and dicarboxylic acid esters (particularly where the composition is a food composition). Other partial fat replacements useful in combination with the fat materials are medium chain triglycerides, highly esterified polyglycerol esters, acetin fats, polyoxyethylene esters, jojoba esters, mono/diglycerides of fatty acids, and mono/diglycerides of short-chain dibasic acids.

Fiber Component

Similarly, food and beverage compositions can be made that combine the present compositions with dietary fibers to achieve the combined benefits of each. By “dietary fiber” is meant complex carbohydrates resistant to digestion by mammalian enzymes, such as the carbohydrates found in plant cell walls and seaweed, and those produced by microbial fermentation. Examples of these complex carbohydrates are brans, celluloses, hemicelluloses, pectins, gums and mucilages, seaweed extract, and biosynthetic gums. Sources of the cellulosic fiber include vegetables, fruits, seeds, cereals, and man-made fibers (for example, by bacterial synthesis). Commercial fibers such as purified plant cellulose, or cellulose flour, can also be used. Naturally occurring fibers include fiber from whole citrus peel, citrus albedo, sugar beets, citrus pulp and vesicle solids, apples, apricots, and watermelon rinds.

These dietary fibers may be in a crude or purified form. The dietary fiber used may be of a single type (e.g., cellulose), a composite dietary fiber (e.g., citrus albedo fiber containing cellulose and pectin), or some combination of fibers (e.g., cellulose and a gum). The fibers can be processed by methods known to the art.

Primarily due to the present compositions, the foods and beverages herein can provide reduced serum cholesterol and thus reduced risk of heart disease. Additionally, the present compositions have acceptable organoleptic properties, particularly flavor and texture, despite the presence of L-arginine, polypeptides thereof, salts thereof, and pro-forms thereof.

Dietary foods can be made with the compositions to meet special dietary needs, for example, of persons who are obese, diabetic, or hypercholesterolemic. The present compositions can be a major part of a low-fat, low-calorie, low-cholesterol diet, and they can be used alone or in combination with drug therapy, nutritional therapy, or other therapy. Combinations of food or beverage products made with the compositions can be used as part of a total dietary management regimen, based on one or more of these products, containing the compositions alone or in combination with one or more of the above-mentioned ingredients, to provide one or more of the above-mentioned benefits.

This discussion of the composition uses, combinations, and benefits, is not intended to be limiting or all-inclusive. It is contemplated that other similar uses and benefits can be found that will fall within the spirit and scope of this invention.

### EXAMPLES

The following examples are illustrative of uses of the present compositions. Such examples are non-limiting illustrations and various modifications thereof may be made by one of ordinary skill in the art with the benefit of the present disclosure.

### EXAMPLE 6

L-arginine is coated under conditions similar to those described herein above. L-arginine in powdered form, and having a particle size of less than about 100 microns is utilized. The L-arginine is coated with a mixture of a sterol and a sterol ester or a mixture of a stanol and a stanol ester. The ratio of sterol/stanol to ester is adjusted to provide a coated material with a malleable form. Sufficient coating is deposited upon the arginine particle to ensure flavor protection. The resulting coated material is mixed into a commercially available peanut butter preparation at a level equivalent to 10 grams of coated material per 100 grams of peanut butter. Two and one half grams of peanut butter, containing approximately 0.175 grams of L-arginine, is applied to two crackers to form a sandwich. The sandwiches are sensory evaluated in the laboratory and exhibit no bitter or fishy off-flavors or aftertaste relative to control peanut butter sandwiches.

### EXAMPLE 7

In a manner similar to that described in Example 6, L-arginine coated with a mixture of sterol and sterol fatty acid ester or stanol and stanol fatty acid ester is added to a cheddar cheese preparation at a level of 10 grams per 75 grams of cheese mixture. Approximately 3 grams of the resulting mixture (containing approximately 0.176 grams of L-arginine) was applied to commercially available crackers. The crackers having the mixture applied thereto are sensory evaluated in the laboratory and exhibit no bitter or fishy off-flavors or aftertaste relative to crackers having a control cheese mixture applied thereto.

### EXAMPLE 8

In a manner similar to that described in Example 6, L-arginine coated with a mixture of sterol and sterol fatty acid ester. A high ratio of sterol relative to sterol ester is used to prepare a coating with excellent thermal stability. The resulting coated L-arginine is added to prepared, commercially available sugar cookie mix at a ratio of 18 grams of coated L-arginine to 510 grams of cookie mix. Eighteen cookies are prepared according to known procedures, each containing approximately 0.5 grams of L-arginine. The resulting cookies retain their natural cookie flavor.

### EXAMPLE 9

A fat-free health bar is prepared having the following composition:

| Component | Wt % |
|---|---|
| Soy Protein Isolates | 28 |
| Fructose | 30 |
| High Fructose Corn Syrup | 23.5 |
| Raisins | 6.8 |
| Coated L-Arginine (Coated as described | 5 |
| herein with a mixture of sterol and sterol fatty | |
| acid ester) | |
| Olean ™ (sucrose polyester, commercially | 6 |
| available from Procter & Gamble Co., | |
| Cincinnati, OH) | |
| Cinnamon | 0.5 |
| Salt | 0.1 |
| Sodium Bicarbonate | 0.1 |

The Sterol ester of L-arginine and Olean™ are pre-mixed prior to blending with the remainder of the dry ingredients and formed into bars. Other dried fruits, for example, cranberries, apricots, and the like may be substituted for the raisins. The health bar is ingested once daily for a period of 12 weeks as a supplement to a normal diet. The health bar is shown to reduce serum cholesterol levels after this 12 week period.

### EXAMPLE 10

A sports energy gel is prepared having the following composition:

| Component | Wt % |
|---|---|
| Maltodextrin | 59 |
| Water | 20 |
| Fructose | 12 |
| Coated L-Arginine (Coated as described | 5 |
| herein with a mixture of sterol and sterol fatty | |
| acid ester) | |
| Citric Acid | 3 |
| Vitamin C | 0.5 |
| Vitamin E | 0.1 |
| Artificial Flavor | 0.2 |
| Sodium Benzoate | 0.1 |
| Potassium Sorbate | 0.1 |

What is claimed is:

1. A composition characterized by:
(a) a first component selected from the group consisting of L-arginine, polypeptides thereof, acceptable salts thereof, pro-forms thereof, and mixtures thereof; and
(b) a second component selected from the group consisting of sterols, stanols, sterol esters, stanol esters, polyol fatty acid polyesters, and mixtures thereof.

2. A composition according to any of the preceding claims wherein the first component is selected from the group consisting of L-arginine and acceptable salts thereof.

3. A composition according to any of the preceding claims wherein the second component is a polyol fatty acid polyester.

4. A composition according to claim 1 or 2 wherein the second component is selected from the group consisting of phytosterols, phytostanols, and fatty acid esters thereof.

5. A composition according to any of the preceding claims comprising from about 0.0001% to about 25% of the first component and from about 0.0001% to about 25% of the second component, by weight of the composition.

6. A composition according to any of the preceding claims comprising from about 0.0001% to about 25% of the first component and from about 0.0001% to about 25% of the second component, by weight of the composition.

7. A composition according to any of the preceding claims comprising from about 1% to about 15% of the first component and from about 1% to about 15% of the second component, by weight of the composition.

8. A kit characterized by a composition according to any of the preceding claims and information wherein the information is selected from the group consisting of:
(a) information that use of the composition provides one or more benefits selected from the group consisting of general health benefits; and
(b) information instructing a treatment regimen for the composition.

9. A kit according to claim 8 wherein the general health benefit is a cardiovascular benefit.

10. A method of promoting a cardiovascular benefit characterized by orally administering to a mammal a composition according to claim 1.
